(19) United States
(12) Patent Application Publication
(10) Pub. No.: US 2018/0160980 A1
(43) Pub. Date: Jun. 14, 2018

(54) SURGICAL POSITIONING DEVICE COVER AND PATIENT RESTRAINT

(71) Applicant: Nue Medical Solutions, LLC, Aurora, IL (US)

(72) Inventors: Aris John Balderama Arenas, Aurora, IL (US); Chris Chung-Ming Huang, Hinsdale, IL (US); Paul M. Korzenecki, Naperville, IL (US)

(21) Appl. No.: 15/823,524

(22) Filed: Nov. 27, 2017

Related U.S. Application Data
(60) Provisional application No. 62/432,533, filed on Dec. 9, 2016.

(51) Int. Cl. A61B 5/00; A61G 13/12

(52) U.S. Cl. CPC A61B 5/702; A61G 13/1295; A61B 46/20; A61B 5/704; A61G 13/129

(57) ABSTRACT

A surgical positioning device cover, kit, and method are provided and are configured for use with a vacuum formable surgical positioning device. The cover includes a main body portion a main opening for receiving the surgical positioning device and at least one pneumatic opening configured to provide access to a valve assembly of the surgical positioning device. The surgical positioning device cover also includes a plurality of straps secured to the main body portion and configured to secure the cover, the surgical positioning device, and a patient to a surgical table.

### CROSS-REFERENCE TO RELATED APPLICATION

This application claims the benefit of and priority to U.S. Provisional Patent Application No. 62/432,533, filed Dec. 9, 2016, the entire contents of which are hereby incorporated by reference.

### BACKGROUND

The present disclosure relates a combination surgical positioning device cover and patient restraint for use in a surgical environment.

Moldable vacuum bean bags are known for positioning patients in a surgical environment. These bean bags are particularly advantageous for supporting a patient on his or her side as frequently utilized, for example, during shoulder surgeries. Moldable vacuum bean bags are generally sized to envelop the patient from approximately the shoulder area to the hip area. The bags are air impermeable and filled with small beads. A valve is provided to introduce or evacuate air from the interior of the bag. When the bag is at least partially inflated the beads are generally free to move within the bag such that the bag can be molded around the patient. When air is removed from the bag the bag compresses the beads and the bag becomes more rigid and maintains its molded shape in order to support the patient

In use, with the valve open and the bag at least partially inflated, the patient is positioned on the bag and the bag is molded around the patient's body. While holding the bag in place the valve is attached to a vacuum source to evacuate the air from the bag. As the air is evacuated the bag becomes semi rigid, as there is no longer room for the beads to move, and the bag is able to support the patient in the desired position.

### SUMMARY

In some aspects, a surgical positioning device cover is provided and comprises a main body portion including a top panel and a bottom panel, the top and bottom panel joined to one another and defining a cavity, the top and bottom panel cooperatively defining a main opening communicating with the cavity and at least one pneumatic opening configured to permit pneumatic communication with the cavity. The surgical positioning device cover also comprises a plurality of straps coupled to the main body portion, the straps including first ends having return members, and second ends having securement members.

In some embodiments, the straps may be substantially parallel to one another. In other embodiments, the main body portion may be substantially rectangular and may include a pair of long edges and a pair of short edges, and the straps may be oriented substantially parallel to the short edges. In still other embodiments, the first and second ends of the straps may extend beyond opposite long edges of the main body portion. Some embodiments are configured such that the first ends extend a first distance away from the main body portion, the second ends extend a second distance away from the main body portion, and the second distance is between about 1.8 times and about 2.2 times the first distance. Other embodiments are configured such that the first ends extend a first distance away from the main body portion, the second ends extend a second distance away from the main body portion, and the first distance is between about 40 percent and about 60 percent of a width of the main body portion.

In some embodiments, each return member may include a ring member, each securement member may include a first portion of the respective second end covered in one of a hook material and a loop material, and a second portion of the respective second end covered in the other of the hook material and the loop material, and the hook material may be securable to the loop material. In other embodiments, the cover may be securable to both a surgical table and a patient by extending each of the second ends through a respective ring member and securing the hook material of each of the second ends to the loop material of the respective second end. In still other embodiments, the straps may be coupled to the bottom panel. Some embodiments are configured such that the straps are coupled to the bottom panel only along a mid-section of the bottom panel, where the mid-section may have a width that is between about 20% and about 40% of a main body portion width.

In other aspects, a surgical positioning kit is provided and comprises a surgical positioning device, the device comprising a substantially air impermeable outer membrane defining a chamber, a plurality of beads filling the chamber, and a valve assembly communicating with the chamber for the introduction and evacuation of air into and from the chamber. The kit may also include one or more embodiments of a surgical positioning device cover as described above, where the surgical positioning device is insertable into a cavity of the surgical positioning device cover via a main opening of the surgical positioning device cover, and where the valve assembly is extendable through a pneumatic opening of the surgical positioning device cover to facilitate the introduction and evacuation of air into and from the chamber.

In some embodiments of the kit, the cover with the surgical positioning device received therein may be securable together to a surgical table via the straps.

In still other aspects, a method of positioning a patient on a surgical table having side rails spaced from a side of the surgical table is provided and comprises inserting a surgical positioning device into a surgical positioning device cover, positioning the cover with the device inserted therein onto the surgical table, extending straps of the cover between the side rails and the side of the surgical table, while the device is inserted in the cover and the cover and device are positioned on the surgical table, positioning the patient on the cover, the device, and the surgical table, forming the device about the patient, evacuating air from the device to render the device substantially rigid, extending the straps of the cover upwardly around an outside of the side rails and at least partially around the patient, and securing ends of the straps to one another to secure both the cover and the device to the surgical table and to secure the patient to each of the cover, the device, and the surgical table.

In some embodiments, inserting the surgical positioning device into the surgical positioning device cover may include inserting the surgical positioning device through a main opening defined by the surgical positioning device cover and positioned at a first end of the surgical positioning device cover. In other embodiments, inserting the surgical positioning device into the surgical positioning device cover may also include extending a valve assembly of the surgical positioning device through a pneumatic opening defined by the surgical positioning device cover and positioned adjacent a second end of the surgical positioning device cover that is opposite the first end. In still other embodiments, the straps may be coupled to a bottom panel of the surgical positioning device cover, and positioning the cover with the device inserted therein onto the surgical table may include orienting the surgical positioning device cover such that the straps are between an upper surface of the surgical table and the bottom panel. Some embodiments may be configured such that forming the device about the patient includes moving portions of the bottom panel away from the upper surface of the surgical table. Other embodiments may be configured such that the straps are coupled to the bottom panel only along a mid-section of the bottom panel having a width that is between about 20% and about 40% of a main body portion width, and where forming the device about the patient includes maintaining the mid-section of the bottom panel against the upper surface of the surgical table. Additional embodiments may be configured such that the straps include first ends including a return member and second ends including a securement member, and where securing ends of the straps to one another includes extending the second ends through a respective return member and closing the securement member.

### BRIEF DESCRIPTION OF THE DRAWINGS

FIG. 1 is perspective view of an exemplary surgical positioning device cover and patient restraint.

FIG. 2 is a perspective view of a top panel of the surgical positioning device cover and patient restraint of FIG. 1.

FIG. 3 is a perspective view of the surgical positioning device cover and patient restraint of FIG. 1 in an inside-out configuration to reveal a stitching pattern.

FIG. 4 is a bottom view of the surgical positioning device cover and patient restraint of FIG. 1.

FIG. 5 is a top perspective view of a kit including the surgical positioning device cover and patient restraint of FIG. 1 and a surgical positioning device inserted therein.

FIG. 6 is a side perspective view of the surgical positioning device cover and patient restraint of FIG. 1 with a surgical positioning device inserted therein.

FIG. 7 is a side perspective view of the surgical positioning device cover and patient restraint of FIG. 1 secured to a surgical table, having a surgical positioning device inserted therein, and securing a patient to both the surgical positioning device and to the surgical table.

FIG. 8 is a section view taken along line 8-8 of FIG. 7.

FIG. 9 is a flow chart depicting a method of securing a patient to a table using the kit of FIG. 5.

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

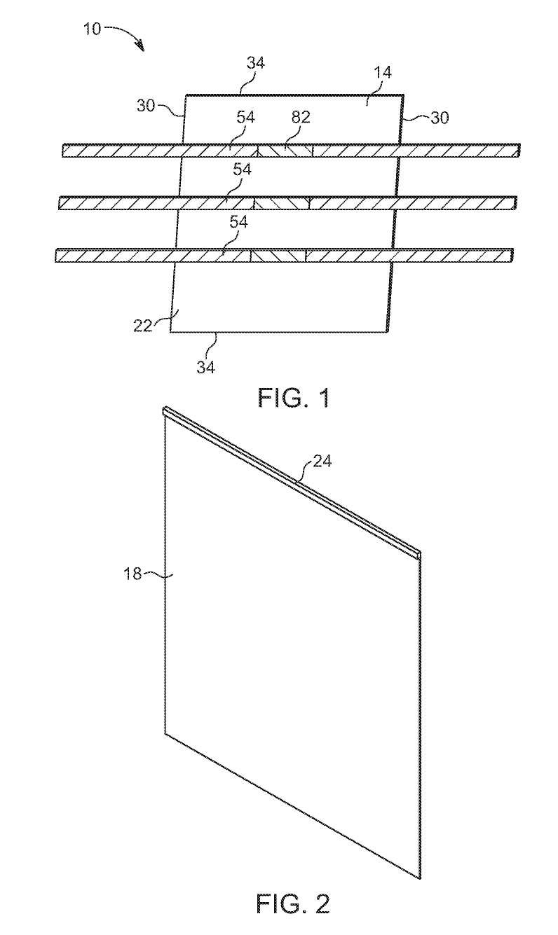

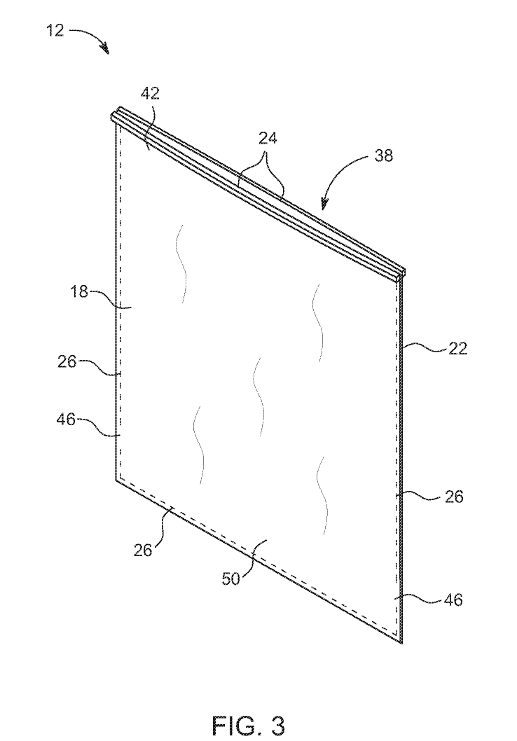

FIGS. 1-4 illustrate a combination surgical positioning device cover and patient restraint (hereinafter the “cover”) 10 according to one exemplary embodiment. The cover 10 includes a main body portion 12 including a top panel 18 (FIG. 2) and a bottom panel 22 (also FIG. 2). In the illustrated embodiment the top and bottom panels 18, 22 are substantially identical. Each top and bottom panel 18, 22 includes a hemmed edge 24, as shown in FIG. 2. As shown in FIG. 3, the top and bottom panels 18, 22 are joined to one another by stitching 26 along their respective perimeters to define the main body portion 12. The main body portion 12 is substantially rectangular and includes a pair of long edges 30 and a pair of short edges 34. The hemmed edges 24 of the top and bottom panels 18, 22 are not stitched to one another. The top and bottom panels 18, 22 are stitched to one another along the long edges 30 and along the short edge 34 that is opposite the hemmed edges 24. When the top and bottom panels are connected to one another the top and bottom panels 18, 22 define a cavity 36 and the hemmed edges 24 define a main opening 38 communicating with the cavity 36. The main opening 38 is positioned at a first end 42 of the main body portion 12.

As shown in FIG. 3, relatively small portions of the top and bottom panels 18, 22 are not stitched to one another along each long edge 30 to define a pair of pneumatic openings 46 communicating with the cavity 36. The pneumatic openings 46 are formed adjacent a second end 50 of the main body portion 12, for reasons discussed below. Although the illustrated configuration includes two pneumatic openings 46, other configurations may include a single pneumatic opening 46 or no pneumatic opening 46 at all. Although the illustrated configuration shows the top and bottom panels 18, 22 joined by stitching, other permanent or semi-permanent joining methods may also be used, including, without limitation, adhesives, sonic welding, hook and loop closures, zippers, and the like. The top and bottom panels 18, 22 may be formed of any of a variety of materials commonly used in surgical environments, including 2 and 3 ply tissue, tissue/polyester blends, polyester/cellulose blends, and non-woven blends. Accordingly, in some embodiments the material of the top and bottom panels 18, 22 may provide both a relatively comfortable outer surface as well as a fluid resistant inner barrier layer.

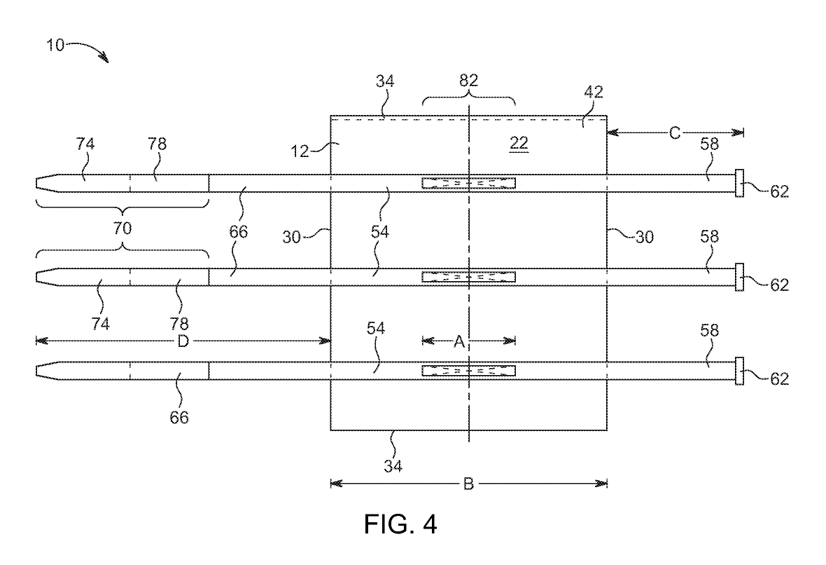

As shown in FIGS. 1 and 4, a plurality of straps 54 are coupled to the bottom panel 22. The straps 54 are oriented to be substantially parallel to one another and also substantially parallel to the short edges 34 of the main body portion 12. Each strap 54 includes a first end 58 having a return member 62 coupled thereto, and a second end 66 having a securement member 70 provided thereon. In the illustrated configuration, each return member 62 includes a ring member, and each securement member 70 includes a first portion 74 of the respective second end 66 covered in one of hook and loop material and a second portion 78 of the respective second end covered in the other of hook and loop material that is securable to the material on the first portion 74. In alternative arrangements, the return member 62 may be eliminated by providing the first and second ends 66 with hook and loop material or another suitable fastening mechanism (snaps, buttons, adhesives, and the like) suitable for securing the first end 58 and the second end 66 to one another.

In the illustrated configuration the straps 54 are coupled to the bottom panel 22 along a mid-section 82 of the bottom panel 22, for example by stitching. By way of example only, the mid-section 82 may have a width A that is between about 20% and about 40% of a main body portion width B. Also in the illustrated configuration, when fully extended in a parallel configuration (e.g., as in FIG. 4), the first and second ends 58, 66 of the straps 54 extend beyond opposite long edges 30 of the main body portion 12. By way of example only, the first end 58 of each strap 54 extends a first distance C away from the main body portion 12, and the second end 66 of each strap 54 extends a second distance D away from the main body portion 12. In some configurations the second distance D may be between about 1.8 times and about 2.2 times the first distance C. Moreover, and by way of example only, in some configurations the first distance C is between about 40 percent and about 60 percent of the main body portion width B. While the illustrated embodiment shows continuous straps 54 extending fully across the width of the main body portion 12, with each strap defining both a first end 58 and a second end 66, alternative embodiments may be configured such that individual straps 54 each defining either a first end 58 or a second end 66 are secured to opposite sides of the main body portion 12.

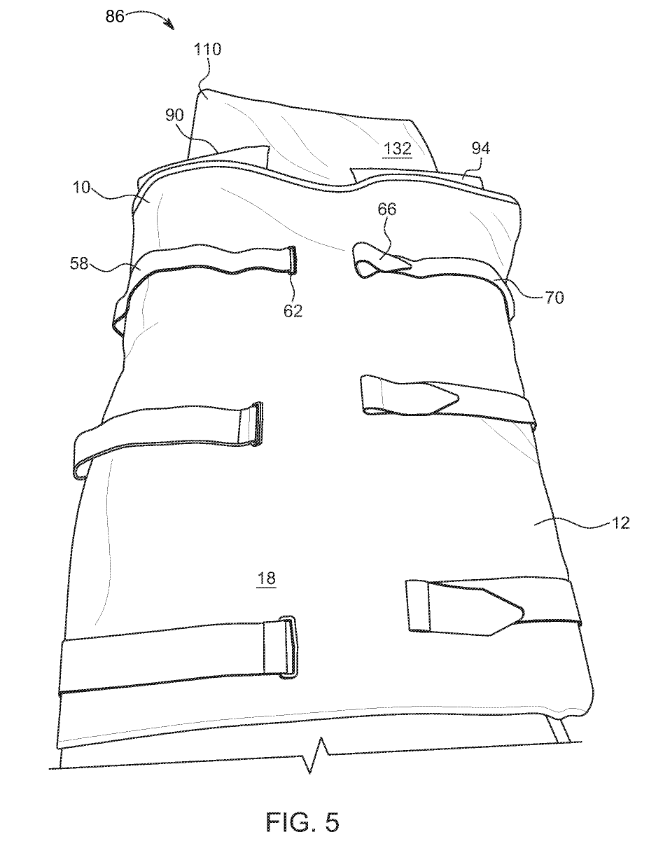

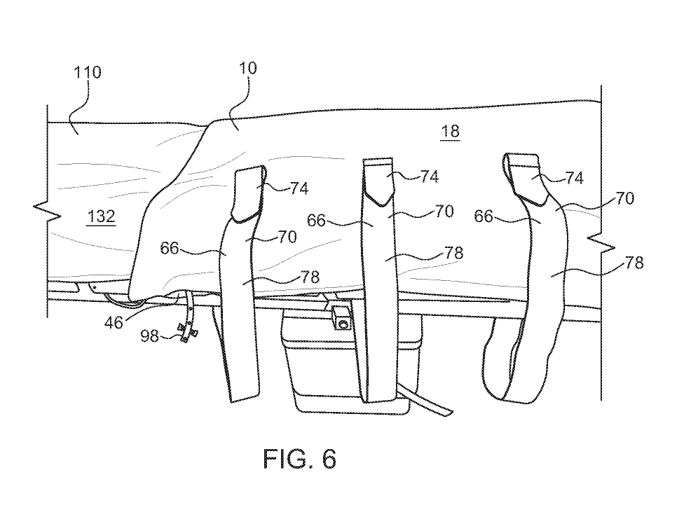

Referring now to FIGS. 5 and 6, a surgical positioning kit 86 includes the cover 10 together with a surgical positioning device 90. In some embodiments, the kit 86 may also include a drape sheet (not shown). The device 90 includes a substantially air impermeable outer membrane 94 and defines a chamber filled with a plurality of beads (not shown). A valve assembly 98 (FIG. 6) communicates with the chamber for the introduction and evacuation of air into and from the chamber in a known manner. The surgical positioning device 90 is insertable into the cavity 36 of the cover 10 via the main opening 38, and the valve assembly 98 is extendable through one of the pneumatic openings 46 to facilitate the introduction and evacuation of air into and from the chamber to render the device 90 formable or substantially rigid, as discussed further below. The surgical positioning device 90 may come in different sizes to accommodate different sized patients. The cover 10 may therefore also come in a variety of sizes corresponding to the different sizes of surgical positioning devices.

The kit 86 may be provided in a common outer packaging, such as a sealed bag, and may include the surgical positioning device 90 together with a plurality of covers 10 and, optionally, a plurality of drape sheets. The covers 10 and the optional drape sheets may be packaged together as sub-kits. Generally speaking, the cover 10 and optional drape sheet are intended to be one-time use items for use with a single patient, after which the cover 10 and optional drape sheet may be discarded and the device 90 may then be inserted into a new cover 10 for use with a subsequent patient. The sub-kits including the cover 10 and optional drape sheet may be provided separately for use after the initial supply of sub-kits has been exhausted or to customers that already have a surgical positioning device 90.

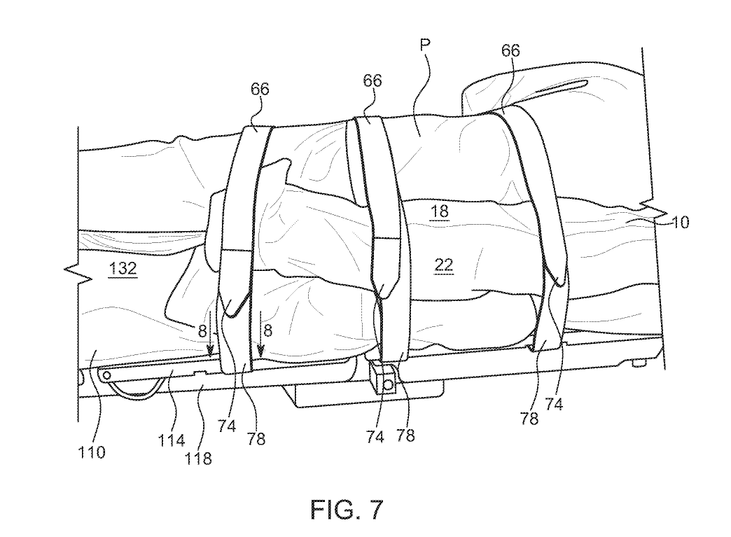

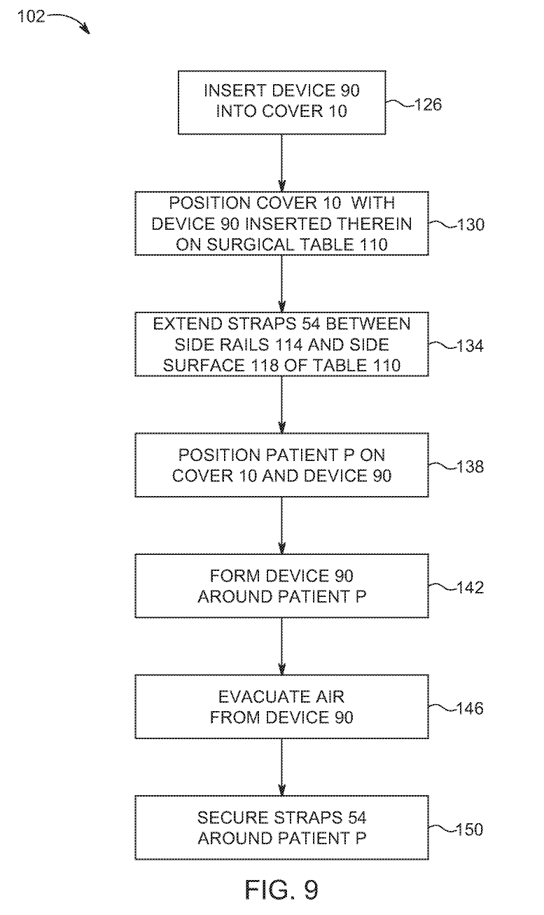

Referring also to FIGS. 7-9, the kit 86, including the cover 10 and the device 90, is configured to provide a method 102 of positioning a patient P on a surgical table 110. The surgical table 110 includes side rails 114 that are spaced from a side surface 118 of the surgical table 110 to define a gap 122. The method 102 includes an inserting step 126 that includes inserting the surgical positioning device 90 into the surgical positioning device cover 10. In some embodiments, the inserting step 126 may include inserting the surgical positioning device 90 through the main opening 38 of the surgical positioning device cover 10, and may also include extending the valve assembly 98 of the surgical positioning device 90 through one of the pneumatic openings 46 so the valve assembly 98 may be accessed from the exterior of the cover 10.

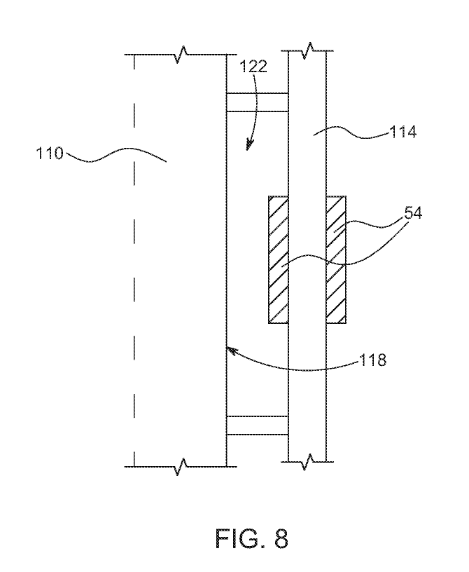

A positioning step 130 may include positioning the cover 10 with the device 90 inserted therein onto the surgical table 110. The positioning step 130 may include orienting the surgical positioning device cover 10 such that the straps 54 are between an upper surface 132 of the surgical table 110 and the bottom panel 22 of the cover 10. An extending step 134 may include extending the straps 54 of the cover 10 generally downwardly between the side rails 114 and the side surface 118 of the surgical table 110. This may be accomplished by extending the first and second ends 58, 66 of the straps through the gap 122 (see FIG. 8) on their respective side of the surgical table 110. A patient positioning step 138 may include positioning a patient P on the cover 10 with the device 90 inserted therein and positioned on the surgical table 110.

The method 102 may then include a forming step 142 that includes forming the device 90 with the cover thereon 10 about the patient P. The forming step 142 may generally include forming the device 90 about the patient P by moving portions of the bottom panel 22 away from the upper surface 132 of the surgical table 110. The forming step 142 also generally includes maintaining the mid-section 82 of the bottom panel 22 against the upper surface 132 of the surgical table 110.

With the device 90 formed around the patient P, an evacuating step 146 may be performed by evacuating air from the device 90 via the valve assembly 98, thereby rendering the device 90 substantially rigid. The evacuating step 146 may be accomplished by connecting the valve assembly 98 to a vacuum source (not shown) that removes the air from the device 90.

A securing step 150 may then be performed by extending the first and second ends 58, 66 of the straps 54 generally upwardly around an outside of the side rails 114 and at least partially around the patient P, and securing first and second ends 58, 66 of the straps 54 to one another. Securing the first and second ends 58, 66 of the straps 54 to one another may include, for example, extending each second end 66 through the return member 62 of its corresponding first end 58, and closing the securement member 70. Closing the securement member 70 may include bringing the first portion 74 of the securement member 70 into contact with the second portion 78 of the securement member 70. With the second end 66 extending through the return member 62 the tightness of the straps 54 may be adjusted by releasing the securement member 70 and letting out or taking in the length of strap 54 that extends through the return member as needed, and then re-closing the securement member 70. While the illustrated examples show each second end extending through the return member 62 of its corresponding first end 58, one or more of the straps 54 may lso be crossed such that the second end of one strap 54 extends through the return member 62 of a different strap 54.

Upon completion of the method 102 discussed above, both the cover 10 and the device 90 are secured to the surgical table 110 by the straps 54. Moreover, the patient P is secured to each of the cover 10, the device 90, and the surgical table 110. As a result, a convenient, simple method of securing the patient, the cover 10, and the device 90 to the surgical table 110 using a common securement, i.e., the straps 54, is provided. Accordingly, the cover 10 is securable to both the surgical table 110 and the patient P by extending the second end 66 of each strap 54 through the corresponding return member 62 (ring member), and securing the first portion 74 (hook material) to the second portion 78 (loop material). The surgical positioning kit 86, e.g., the cover 10 and the surgical positioning device 90 are securable to the surgical table 110 via the straps 54.

Various features of the invention are set forth in the following claims.

What is claimed is:

1. A surgical positioning device cover comprising:
a main body portion including a top panel and a bottom panel, the top and bottom panel joined to one another and defining a cavity, the top and bottom panel cooperatively defining a main opening communicating with the cavity and at least one pneumatic opening configured to permit pneumatic communication with the cavity; and
a plurality of straps coupled to the main body portion, the straps including first ends having return members, and second ends having securement members.

2. The cover of claim 1, wherein the straps are substantially parallel to one another.

3. The cover of claim 2, wherein the main body portion is substantially rectangular and includes a pair of long edges and a pair of short edges, and wherein the straps are oriented substantially parallel to the short edges.

4. The cover of claim 3, wherein the first and second ends of the straps extend beyond opposite long edges of the main body portion.

5. The cover of claim 4, wherein the first ends extend a first distance away from the main body portion, wherein the second ends extend a second distance away from the main body portion, and wherein the second distance is between about 1.8 times and about 2.2 times the first distance.

6. The cover of claim 4, wherein the first ends extend a first distance away from the main body portion, wherein the second ends extend a second distance away from the main body portion, and wherein the first distance is between about 40 percent and about 60 percent of a width of the main body portion.

7. The cover of claim 1, wherein each return member includes a ring member, wherein each securement member includes a first portion of the respective second end covered in one of a hook material and a loop material, and a second portion of the respective second end covered in the other of the hook material and the loop material, and wherein the hook material is securable to the loop material.

8. The cover of claim 7, wherein the cover is securable to both a surgical table and a patient by extending each of the second ends through a respective ring member and securing the hook material of each of the second ends to the loop material of the respective second end.

9. The cover of claim 1, wherein the straps are coupled to the bottom panel.

10. The cover of claim 9, wherein the straps are coupled to the bottom panel only along a mid-section of the bottom panel having a width that is between about 20% and about 40% of a main body portion width.

11. A surgical positioning kit comprising: a surgical positioning device, the device comprising a substantially air impermeable outer membrane defining chamber, a plurality of beads filling the chamber, and a valve assembly communicating with the chamber for the introduction and evacuation of air into and from the chamber; and
the cover of claim 1, wherein the surgical positioning device is insertable into the cavity via the main opening, and wherein the valve assembly is extendable through the pneumatic opening to facilitate the introduction and evacuation of air into and from the chamber.

12. The kit of claim 11, wherein the cover with the surgical positioning device received therein are securable together to a surgical table via the straps.

13. A method of positioning a patient on a surgical table, the surgical table including side rails spaced from a side of the surgical table, the method comprising:
inserting a surgical positioning device into a surgical positioning device cover;
positioning the cover with the device inserted therein onto the surgical table;
extending straps of the cover between the side rails and the side of the surgical table;
while the device is inserted in the cover and the cover and device are positioned on the surgical table, positioning the patient on the cover, the device, and the surgical table;
forming the device about the patient;
evacuating air from the device to render the device substantially rigid;
extending the straps of the cover upwardly around an outside of the side rails and at least partially around the patient; and
securing ends of the straps to one another to secure both the cover and the device to the surgical table and to secure the patient to each of the cover, the device, and the surgical table.

14. The method of claim 13, wherein inserting the surgical positioning device into the surgical positioning device cover includes inserting the surgical positioning device through a main opening defined by the surgical positioning device cover and positioned at a first end of the surgical positioning device cover.

15. The method of claim 14, wherein inserting the surgical positioning device into the surgical positioning device cover includes extending a valve assembly of the surgical positioning device through a pneumatic opening defined by the surgical positioning device cover and positioned adjacent a second end of the surgical positioning device cover that is opposite the first end.

16. The method of claim 13, wherein the straps are coupled to a bottom panel of the surgical positioning device cover, and wherein positioning the cover with the device inserted therein onto the surgical table includes orienting the surgical positioning device cover such that the straps are between an upper surface of the surgical table and the bottom panel.

17. The method of claim 16, wherein forming the device about the patient includes moving portions of the bottom panel away from the upper surface of the surgical table.

18. The method of claim 17, wherein the straps are coupled to the bottom panel only along a mid-section of the bottom panel having a width that is between about 20% and about 40% of a main body portion width, and wherein forming the device about the patient includes maintaining the mid-section of the bottom panel against the upper surface of the surgical table.

19. The method of claim 13, wherein the straps include first ends including a return member and second ends including a securement member, and wherein securing ends of the straps to one another includes extending the second ends through a respective return member and closing the securement member.
